(19) United States
(12) Patent Application Publication
(10) Pub. No.: US 2008/0261229 A1
(43) Pub. Date: Oct. 23, 2008

(54) SIMULTANEOUS RAPID DETECTION OF MICROBES

(76) Inventors: Angela M. Oppedahl, Boone, IA (US); Steven J. Lasky, Ankeny, IA (US); Dylan D. Baker, West Des Moines, IA (US); Daniel A. Buttry, Laramie, WY (US); Ann M. Steger, Des Moines, IA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 12/103,282

(22) Filed: Apr. 15, 2008

Related U.S. Application Data
(63) Continuation-in-part of application No. 11/729,120, filed on Mar. 28, 2007.
(60) Provisional application No. 60/786,498, filed on Mar. 28, 2006.

(51) Int. Cl. C12Q 1/68; C12Q 1/04

(52) U.S. Cl. 435/6; 435/34

(57) ABSTRACT

The invention includes a method of simultaneously detecting the presence or absence of more than one microbe class (e.g., bacteria, yeast, and mold) in a sample. The method can include the step of applying a sample suspected of containing more than one microbe class to a growth medium, optionally fragmenting the sample, associating the sample with a labeling agent, and simultaneously detecting the presence or absence of each microbe class by detection of the labeling agent.

### RELATED APPLICATIONS

This application is a continuation-in-part of U.S. patent application Ser. No. 11/729,120, titled Rapid Detection of Mold by Accelerated Growth and Detection, filed Mar. 28, 2007, which itself claims the benefit of U.S. Provisional Patent Application Ser. No. 60/786,498, titled Rapid Detection of Mold by Accelerated Growth and Detection, filed Mar. 28, 2006, the contents of each of which are hereby incorporated by reference.

### FIELD OF THE INVENTION

The invention generally relates to the detection of microbes in a sample.

### BACKGROUND OF THE INVENTION

For many products, determining if the product contains microbes is an important consideration before the product is placed on the market. Such considerations are particularly important for products intended to be used on or in a mammalian body. Unfortunately, many current methods of testing for the presence of microbes take several days to complete. Further, many approaches require different sample preparation and detection methods for different microbe classes, such as bacteria, yeast, and mold.

### SUMMARY OF THE INVENTION

In some embodiments, the invention includes a method of simultaneously detecting the presence or absence of different microbe classes (e.g., bacteria, yeast, and mold) in a sample. By “simultaneous,” it is meant that two or more of the microbe classes can be detected using a single sample preparation and detection method. The method can include the step of applying a sample suspected of containing microbes to a growth medium. After the sample is exposed to the growth medium for a sufficient duration to grow microbes present in the sample, the sample can optionally be fragmented. After fragmentation, the sample can be associated with a labeling agent, and the presence or absence of the microbe classes in the sample can be determined by detecting the microbes that are associated with the labeling agent. Such embodiments of the invention accelerate the growth of molds in the sample, and significantly reduce the time to detection compared to traditional tests. Further, such embodiments can also simultaneously detect more than one microbe class present in the sample. For example, in some embodiments of the invention, the presence or absence of the different microbe classes in a sample can be determined in less than about twenty-four hours.

Applicant has discovered the manner in which a single sample can be obtained and used in a manner sufficient to determine the presence or absence of bacteria, yeast and/or mold in the sample, simultaneously and within a short period of time. This can be compared, for instance, to methods in which the growth conditions suitable for one or the other of these microbial classes may not be useful for the others, as well as situations in which the assessment of these various classes within a single sample might require the use of different sample aliquots and/or test methods.

In some embodiments, the invention includes a method for detecting the presence or absence of microbes in a sample. Such embodiments may include the steps of applying a sample suspected of containing microbes to a growth medium having a substrate useful for supporting bacterial, yeast and mold growth, associating the sample with a labeling agent, the labeling agent including a fluorescent agent, and simultaneously detecting the presence or absence of bacteria, yeast, and mold in the sample by placing the substrate into a fluorescence chamber, the presence of bacteria, yeast and mold being detectable by the labeling agent(s) associated with the respective bacteria, yeast, and mold. In some embodiments, only a single labeling agent is utilized to detect the bacteria, yeast, and mold, and the bacteria, yeast, and mold are differentiated by their respective locations on a scatter plot.

### BRIEF DESCRIPTION OF THE DRAWINGS

FIG. 1 shows a scatter plot output of a sample discussed in Example 1.

FIG. 2 shows a scatter plot output of a mold spiked sample discussed in Example 1.

FIG. 3 shows a scatter plot output of a bacteria spiked sample discussed in Example 1.

FIG. 4 shows a scatter plot output of a yeast spiked sample discussed in Example 1.

FIG. 5 shows a scatter plot output of a sample discussed in Example 2.

FIG. 6 shows a scatter plot output of a spiked sample discussed in Example 2.

### DETAILED DESCRIPTION OF THE INVENTION

For the purpose of promoting an understanding of the principles of the invention, reference will now be made to the embodiments illustrated in the drawings and specific language will be used to describe the same. It will, nevertheless, be understood that no limitation of the scope of the invention is thereby intended; any alterations and further modifications of the described or illustrated embodiments, and any further applications of the principles of the invention as illustrated therein, are contemplated as would normally occur to one skilled in the art to which the invention relates.

Embodiments of the invention relate to a method of simultaneously detecting more than one eucaryotic and/or procaryotic microbe class (e.g., bacteria, yeast, and mold) in a sample. In some embodiments, the method includes the steps of preparing the sample, applying the sample to a growth medium, growing the sample on or in the presence of the growth medium, fragmenting the sample, associating the sample with a labeling agent, and simultaneously detecting the presence or absence (in either qualitative and/or quantitative terms) of more than one microbe class in the sample. Such a method is useful for rapidly detecting the presence or absence of microbes in a sample without having to run separate tests for bacteria, yeast, and mold. Generally, many products can be tested for the presence of absence of the microbe classes in less than about 24 hours. Depending on product sample composition, some products may take up to 48 hours, and in some cases, more, to test.

A wide variety of samples can be analyzed to determine if microbes are present or absent in accordance with embodiments of the invention. For example, samples of commercial products (e.g., over-the-counter products, household products and/or oral care products) can be tested to determine if they contain microbes before they are placed in commerce. Examples of such commercial products include products designed or intended for application or use on or in a mammalian body. In some embodiments, the sample can include a personal care product (PCP); for example, make-up (such as eye shadow, blush, mascara or the like); over-the-counter medications (such as nasal spray, vitamins, aspirin or the like); beauty products (such as shampoo, lotion, shaving gels/creams) or any product that may be used as a personal care item. In some embodiments, the invention relates to a method for preparing PCP samples for a compendial methods test.

Accordingly, methods in accordance with embodiments of the invention are useful for allowing a sample of a commercial product to be tested for the presence or absence of microbes and providing a rapid determination of whether microbes are present in the sample without having to run separate tests for different microbe classes. Such a rapid determination is useful, for example, because it allows the batch of the commercial product from which the sample was taken to be released into the market if the sample passes the microbe presence/absence test or, in the alternative, disposed of or treated if the sample fails the microbe presence/absence test. Such a determination can be made in a relatively quick manner that avoids the need for storing the batch of commercial product for long periods of time.

Embodiments of methods of the invention are useful for simultaneously detecting various types of bacteria, yeast, and mold. For example, bacteria detectable by the method includes spirochetes, aerobic and anaerobic organisms, gram negative or gram positive bacteria, including those found in the form of rods, cocci, and spores, yeast detectable by the method includes unicellular and budding yeast, and mold detectable by the method include spores and filamentous mold.

Representative bacteria are selected from the group consisting of the following genera Aeromonas (e.g., hydrophila), Alcaligenes (e.g., faecalis and viscolactis), Aspergillus (e.g., niger), Bacillus (e.g., cereus, megaterium, subtilis, and stearothermophilus), Branhamella (e.g., catarrhalis), Corynebacterium (e.g., xerosis), Enterobacter (e.g., aerogenes), Enterococcus (e.g., faecalis), Escherichia (e.g., coli), Flavobacterium (e.g., capsulatum), Klebsiella (e.g., pneumoniae), Lactobacillus (e.g., plantarum), Lactococcus (e.g., lactis), Leuconostoc (e.g., mesenteroides), Micrococcus (e.g., luteus and roseus), Mycobacterium (e.g., smegmatis), Proteus (e.g., mirabilis and vulgaris), Pseudomonas (e.g., aeruginosa and fluorescens), Rhodospirillum (e.g., rubrum), Saccharomyces (e.g., cerevisiae), Salmonella (e.g., typhimurium), Serratia (e.g., marcescens), Staphylococcus (e.g., aureus and epidermidis), and Streptococcus (e.g., bovis and mitis).

Representative yeast are selected from the group consisting of Saccharomyces (e.g., cerevisiae), Schizosaccharomyces (e.g., pombe) and Candida (e.g., albicans).

Representative molds are selected from the group consisting of the genera Acremonium, Alternaria, Arthrinium, Ascospores, Aspergillus, Aureobasidium, Basidiospores, Beauveria, Botrytis, Chaetomium, Chrysonilia, Curvularia, Drechslera, Exophiala, Fusarium, Geotrichum, Gliocladium, Memnoniella, Mucor, Myxomycete, Paecilomyces, Penicillium, Phoma, Pithomyces, Rhizopus, Scopulariopsis, Stachybotrys, Syncephalastrum, Taeniolella, Trichoderma, Torula, Ulocladium, Verticillium, and Zygomycetes, and various species thereof.

The sample can be prepared by any suitable method. In some embodiments, the sample can be directly taken from a batch of a commercial product. The sample size of the product should be sufficient to provide an adequate sample, regardless of the sampling procedure. In some embodiments, the sample of the commercial product is subjected to one or more preparation steps before it is applied to a growth medium. For example, various anti-microbial agents present in the sample can be neutralized so that they do not interfere with the growth of any microbes present in the sample. In certain embodiments, the sample preparation includes a dilution step where about 10 grams of the product is placed in about 90 mL of a media (such as those indicated in applicable compendial guidelines) or buffer (such as phosphate buffer). After dilution, the sample can be placed in a growth enhancement medium. In some embodiments the sample can be diluted anywhere from 1:1 to 1:100 or more with media, and preferably about 1:10.

The growth enhancement medium can include a variety of media useful for growing microbes, especially, bacteria, yeast and mold, present in the sample. Growth media can be prepared and used in any desirable manner. See, for instance, United States Pharmacopeia (USP) Chapter 61, USP30-NF25, and/or the Difco & BBL Manual: Manual of Microbiological Culture Media, 2003, the disclosures of each of which are incorporated herein by reference.

Suitable media can be provided in any suitable manner, e.g., as nutrient media, including undefined media (also known as basal or complex media) and defined nutrient media, as well as differential medium, minimal media, selective media, transport media, and enriched media.

In some embodiments, the growth medium is useful for facilitating the rapid growth of several microbe classes present in the sample, that are easy to fragment in accordance with embodiments of the invention, as discussed further below, and/or does not interfere with the microbe detection step of embodiments of the invention, as described further below. In some embodiments, the growth medium is useful for stimulating bacteria, yeast and mold growth and producing mold hyphae. An array of media can be used, e.g., as described in USP Chapter 61, incorporated by reference above.

For example, the growth medium may comprise a nitrogenous substance useful for promoting fungal growth, such as Tryptic Soy Broth (e.g., at a concentration of about 5 g/L to about 30 g/L (e.g., about 30 g/L)); a sample neutralization agent, such as soy lecithin (e.g., at a concentration of about 5 g/L to about 50 g/L (e.g., about 5 g/L)) and/or Tween-20 (e.g., at a concentration of about 40 mL/L to about 100 mL/L (e.g., about 40 mL/L)); a substance high in carbohydrate content, such as malt extract (e.g., at a concentration of about 5 g/L to about 20 g/L (e.g., about 10 g/L)); and/or a carbon source, such as dextrose (e.g., at a concentration of about 2 g/L to about 20 g/L (e.g., about 10 g/L)).

In some embodiments, the growth medium includes a stimulation agent to stimulate growth of the hyphal strands. The stimulation agent may also stimulate the growth of yeast and/or bacteria. In certain embodiments, the stimulation agent is n-acetyl glucosamine. N-acetyl glucosamine (NAG) is a naturally occurring amino sugar that is a primary component of chitin, the fibrous structure of the fungal cell wall. The n-acetyl glucosamine can be included at any concentration suitable to stimulate the growth of the hyphal strands. For example, the n-acetyl glucosamine can be present at a concentration range of about 5 mM to about 450 mM (e.g., about 50 mM to about 100 mM, such as about 75 mM). In certain embodiments, the growth medium is a liquid (sometimes referred to as a broth).

In some embodiments, the growth medium is made as follows: The nitrogenous substance useful for fungal growth, the sample neutralization agent, the substance high in carbohydrate content, and/or the carbon source can be measured and/or weighed and placed in about 1 L of deionized water. In some embodiments, the carbon source can be added in a later step. This solution can be mixed with a magnetic stir bar while heated to a boiling temperature to dissolve the ingredients. The solution can then be autoclaved at about 121° C. for about 15-60 minutes, cooled to room temperature and the growth stimulation agent (and, in some embodiments, the carbon source) can be weighed and added to the mixture while stirring. The broth pH can then be measured and adjusted to 7.0±0.2. The entire solution can then be sterile filtered through a 0.2 um filter. In certain embodiments, each component could be measured and/or weighed, boiled to dissolve, pH adjusted, sterile filtered and/or autoclaved for sterility. The growth medium can be stored at a storage temperature of about 4° C.±2° C., and should be stable for at least about 6 months.

In some embodiments, the growth medium can include one or more solid articles (including but not limited to substrates, such as, swabs, mesh-like devices attached to a shaft, sponges, and/or free-standing meshes or screens, and/or fragmentation enhancers, such as small bead-like objects, metallic beads, ceramic beads, chips of ceramic materials, magnets, and the like). The solid articles can provide a place for microbes, especially mold, to attach and grow, and/or can be useful for fragmenting microbes, such as mold, as described further below.

In some embodiments, a solid article comprising a substrate can be utilized to concentrate and isolate the microbes, especially mold, from the product matrix and other debris in the sample. The substrate can be placed on or in the growth medium and, in some embodiments, can be removed after microbe growth is promoted (e.g., less than or about 24-48 hours). In certain embodiments, the substrate is porous and supports growth of the microbes onto its porous surface.

The substrate can include any characteristic useful for supporting microbe growth. For example, the substrate can include one or more of a swab, mesh-like device attached to a shaft, and/or free-standing meshes or screens, sponge, and/or combinations thereof. In some embodiments, the substrate comprises a material selected from the group consisting of polyester, polyurethane, cotton, foam, rayon, polyether or any combination thereof. In some embodiments, the substrate can be coupled to a shaft that is useful for allowing a technician to remove the substrate from the growth medium. The shaft of the swab can include any suitable material, such as polypropylene, wood, plastic, metal or any combination thereof. In some embodiments, the substrate is adhered to the bottom of a tube containing the growth medium. The growth medium and sample can be poured off after sufficient contact time with the substrate.

After microbe growth has been adequately promoted, the substrate can be removed from the growth medium and placed in a buffer solution to concentrate the microbe in a smaller volume and remove it from the product matrix. In embodiments where mold is primarily concentrated on the substrate, the transfer of the substrate and the residual growth medium contained therein will also transfer bacteria and yeast. It can be useful to remove the microbes from the product matrix because the product matrix, in many samples, including PCP samples, often include extraneous materials that can interfere with accurate mold detection during the detection step (e.g., by contributing to background counts), increasing the chance for a false positive result. The material being tested will be continued to be referred to as the “sample” herein, although the product matrix has been removed.

The buffer solution can include phosphates and may include salt. The buffer solution is useful for the reactionary phase of the fluorescent dyes used in some embodiments of the invention, and helps promote the stability of the sample. The buffer solution can also contain a chemical fragmenting agent to fragment the mold hyphae, as discussed further below. The buffer solution can also contain an anti-foaming agent, such as antifoam A, and/or antifoam 204 or the like. This is useful to suppress the formation of foam during any physical agitation of the sample, such as during mechanical fragmenting as discussed further herein.

In some embodiments, the sample is fragmented. Such fragmentation can be useful when the sample is subjected to a detection step, as described further below. The fragmenting step can be any method of causing any mold present within the sample to fragment. For example, the fragmentation can include disassociation of mold hyphae into individual segments. In some embodiments, the fragmenting is promoted by a chemical fragmenting agent and/or a mechanical fragmentation step.

As mentioned above, a chemical fragmenting agent can be used to break the hyphal segments apart and into individual objects that can be detected, such as by exposing the hyphal segments to a solution containing a chemical fragmenting agent. In some embodiments, the chemical fragmenting agent includes one or more of the following: organic acids (including, but not limited to, organic alkyl and aryl acids such as hexanoic, benzoic, and/or lactic acid); inorganic acids (including, but not limited to, sulfuric acid, perchloric acid, hydrochloric acid, and/or methane sulfonic acid); inorganic bases (including, but not limited to, NaOH, KOH and/or other sources of hydroxide ion or high pH conditions); oxidizing agents (including, but not limited to, H₂O₂, hypochlorite, and/or nitric acid); cationic agents (including, but not limited to, CTAB, dodecyltrimethyl ammonium salts, quaternized pyridinium salts and the like); surfactants (including but not limited to, Tween-20, Triton X-100, PEG and the like); alcohols (including, but not limited to, ethanol, isopropanol and the like); and/or enzymes that attack cells walls (including, but not limited to, beta-glucanase, chitinase, protease, and/or cellulase). In some embodiments, the chemical fragmenting agent includes acetic acid. The chemical fragmenting agent can be included in a solution in any concentration range useful for fragmenting the mold in the sample. For example the chemical fragmenting agent can be in a concentration range of about 100 nM to about 5M (i.e., about 0.01% v/v to about 25% v/v). In some embodiments, the concentration is about 80 mM to about 4.16 M (i.e., about 0.5% v/v to about 25% v/v).

Also as mentioned above, some embodiments of the invention include a mechanical fragmentation step to fragment the sample. The mechanical fragmentation step can include any process useful for fragmenting the sample. In some embodiments, the mechanical fragmentation step includes sonic, high shear force, or other methods to disrupt the connections between the hyphae segments; mechanical agitation of a fluid containing the mold using any of a variety of devices (including, but not limited to, vortexers, shakers, mixers, magnetic stir bars, homogenizers, pulverizers and the like); mechanical agitation of the fluid containing the mold and also containing small bead-like objects that assist in disrupting the connections between the hyphae segments (including but not limited to, ceramic beads, stainless steel beads, glass beads, small mineral or other inorganic particles, such as metal oxides, and the like); exposure to sonic energy (including, but not limited to, sonic cell disruptors, probe sonicators, ultrasonic cleaning baths and the like); and/or forcing the fluid containing the mold through a small orifice at a high flow velocity so as to generate large shear forces in such a way as to cause disruption of the connections between the hyphae segments using any of a variety of apparatuses (including but not limited to, syringes, fluid jets, counter-rotating coaxial cylindrical chambers and the like). Any of these mechanical fragmentation steps can be performed in lieu of or in addition to utilization of any of the chemical fragmenting agents described above. Further, a solid article (e.g., small bead-like objects, metallic beads, ceramic beads, chips of ceramic materials, magnets, and the like) can also be utilized with any of these processes and/or devices to fragment the mold, such as by grinding or pulverizing the mold within and on the side of a sample tube.

In some embodiments, any solid article (e.g., substrate useful for growing microbes) present is removed from the process before the mold is fragmented. For example, in some embodiments the mold is extracted from the solid article in the buffer solution. In other embodiments, the solid article is subjected to the fragmenting step and removed from the sample after the fragmenting step. In either embodiment, a portion of the solid article can be present in the microbe detection step. For example, as mold grows enzymes are released that act to enhance the spread of mold (e.g., mold that grows on wood releases cellulase that breaks down cellulose in the wood grain). This allows the mold to attach and penetrate this substrate for growth. A similar phenomenon may also occur on the solid article, and when the sample is subjected to fragmentation these affected areas can ‘shed’ the solid article as well as the mold. In some embodiments, as discussed further below, the substrate is not removed from the sample before the detection step.

After fragmentation, the sample can be filtered. Filtering can be useful, for example, for removing larger pieces of a substrate from the sample. Such larger pieces may interfere with microbe detection. In some embodiments, the sample is passed through a filter with a screen size of about 30 to about 40 microns (e.g., about 35 microns). In some embodiments, the filter is included in a tube cap, and the sample is filtered as it is delivered (e.g., via a pipettor) through the filter into the tube. In such embodiments, it is useful to keep the pipettor upright with its tip touching the filter to help ensure the desired volume of sample passes through the filter. In certain embodiments, the tube can contain buffer solution and specific amounts of the sample can be added to the tube through the filter to achieve a desired ratio of sample to solution.

In some embodiments, the fragmented sample is labeled with a labeling agent. The labeling agent can be any agent capable of associating itself with any microbes present in the sample which can be detected in a detection step. The labeling agent can be used as a proxy for detecting the presence or absence of the microbes in the detection step. Examples of labeling agents include fluorescent agents and luminescent agents which can be detected by fluorescence and luminescence, respectively.

In some embodiments, fluorophores are introduced to the sample, such as by fluorescent labeling of the microbes, and the presence of the microbes can be detected from these labeled segments using fluorescence. In other embodiments, a luminescent agent, such as luciferase or adenylate kinase, is introduced to the sample and the presence or absence of microbes in the sample can be detected by adenosine triphosphate (ATP)-induced luminescence.

In certain embodiments, following fragmentation of the mold hyphae into smaller units, they and the rest of the microbes are exposed to a fluorescent labeling agent. The fluorescent agent can include any agent useful for fluorescently labeling the sample. In some embodiments, the fluorescent agent includes nucleic acid dyes/stains, fluorochrome labeled anti-mold (component) antibodies, mold specific stains, and/or fluorochrome labeled nucleic acid probes. The fluorescent labeling agent may include one or more nucleic acid dyes/stains (including, but not limited to, those available under the tradenames Syto 62 (Invitrogen, Cat. # S11344), Syto 61 (Invitrogen, Cat. # S11343), Syto 59 (Invitrogen, Cat. # S11340), Hexidium Iodide (Invitrogen, Cat. # H7593) or the like); mold specific stains (including, but not limited to, Calcofluor white (Sigma, Cat. #18909), Fluorescent Brightener 28 (Sigma, Cat. # F3543) or the like); or fluorescent stains (including, but not limited to, trypan blue (Sigma, Cat. #302643), aniline blue (Sigma, Cat. #415049), congo red (Sigma, Cat. # C6277) and the like); fluorochrome labeled antibodies or probes (fluorochromes, including, but not limited to, Cy5 (Pharmacia, Cat. # PA15000), Alexa 647 (Invitrogen, Cat. # A20186), WellRed dyes (IDT Technologies), Oyster family dyes (IDT Technologies), Evoblue30 (Sigma, Cat. #49328) or the like). The stains/dyes generally stain external and internal mold components, the fluorochrome labeled antibodies generally target external distinctive epitopes unique to that mold strain, and the fluorochrome labeled probes generally target the DNA/RNA of the internal mold structure.

Various cell permeant stains, including fluorescent stains, can be used for yeast and bacteria, as well as molds as described above. See, e.g., Conn's Biological Stains: A Handbook of Dyes, Stains and Fluorochromes for Use in Biology and Medicine, 2002, the disclosure of which is incorporated herein by reference.

In some embodiments, the fluorescent agent includes immobilized fluorophores. Such immobilized fluorophores can be immobilized on a surface in contact with the samples, such as latex spheres, magnetized spheres, and nanoparticles.

In some embodiments, a quenching agent can be applied to the fluorescently labeled sample to block non-target labeling. Such a quenching agent is useful for the quenching of non-target segments. For example, Acid Black (e.g., Acid Black 48), Irgalon, Acid Blue or the like can be used as a quenching agent.

After association with fluorophore, the presence or absence of the different microbe classes can be simultaneously detected by detection of the fluorophore. In some embodiments, detection of the individual fluorescently labeled microbes is achieved using flow cytometry.

In some embodiments, a flow cytometer includes a housing and a sample inlet adapted to allow a sample to be injected into the flow cytometer. The flow cytometer also includes a light wave source (e.g., a laser source) for passing light through the sample. The flow cytometer also can include electronics for sensing, measuring, manipulating and reporting the fluorescence of the sample as it passes through the light source. Further, the flow cytometer can include electronics for sensing, measuring, manipulating, and reporting a light scattering signal produced as the sample is passed through the light source. An example of a flow cytometer useful for utilization with embodiments of the invention is the Micro PRO™, produced by Advanced Analytical Technologies, Inc., assignee of the present application.

In embodiments utilizing a flow cytometer for simultaneous detection of the microbe classes, the sample is placed into the flow cytometer. Detection is achieved by measurement of the fluorescence and scattering signals that are generated as the microbes pass through a laser beam that intersects the sample path. The flow cytometry measurement can be triggered by either the fluorescence or scatter signal. Upon triggering, both measurements (fluorescence and scatter) can be captured by electronics. The population of each microbe class (e.g., bacteria, yeast, and mold) is identified by virtue of its location on the scatter plot. Because of its unique and reproducible position in the scatter plot, each microbe class can generally be distinguished from each other and other materials in the sample (i.e., the identification of the different microbe classes is generally discriminate), as long as the bacteria population is relatively small. If the bacteria population is so large that it expands into the mold and yeast areas, mold and/or yeast may not be discriminately detectable. However, such a sample would still fail the microbe presence/absence test because of the large bacteria population.

For example, the method can be performed on known negative product samples and analyzed on the flow cytometer. The output of the flow cytometer, such as an intensity plot, establishes a baseline for that particular product matrix. The negative samples have little or no microbes but may have background counts in one or more of the predefined areas for bacteria, yeast, and mold in the scatter plot output due to the product matrix. Once the baseline has been established, all test samples tested in this product matrix can be divided against the baseline to establish a signal to noise ratio. Each predefined area for bacteria, yeast, and mold of the scatter plot output can be compared to its corresponding area in the baseline scatter plot output to simultaneously and generally discriminatively determine the presence or absence of bacteria, yeast, and mold in the sample. For example, signal to noise ratios greater than or equal to a predetermined multiple or ratio above the baseline in each microbe class can be considered positive (“fail”) in the each microbe presence/absence test. There may also be an ambiguous range that falls below the positive range and above the negative range. A positive to negative ratio that falls below the ambiguous range would be considered negative (“pass”).

In some embodiments, the method of the invention does not include a fragmentation step. In such embodiments, microbes are left on a substrate used to support microbe growth. The fluorescent labeling steps described above are then performed by exposing the substrate to a solution containing the labeling agent. Unwanted fluorescence can be quenched by exposing the substrate to a solution containing a quenching agent. The presence of microbes can be detected by placing the entire substrate into a fluorescence chamber such that the presence of microbes can be detected by the fluorescence of the label attached to the microbes. In certain embodiments, the quenching agent may be added directly to the fluorescence chamber, as, for example, in the case in which the substrate is present in the chamber immersed in a solution containing the quencher.

Accordingly, embodiments of the invention include a method of simultaneously detecting the presence or absence of more than one microbe class in a sample that significantly reduces the time to detection compared to traditional tests and allows for a presence/absence determination of bacteria, yeast, and mold in one test.

### EXAMPLES

The following examples are presented for illustrative purposes and are not intended to limit the scope of the claims that follow.

Generally, after product neutralization (i.e., the neutralization of any antimicrobial agents present in the product), the sample is placed in a growth medium that contains a substrate (e.g., swab). This medium stimulates microbe growth, especially, hyphal growth in the mold life cycle to a detectable level within 24 hours for most samples and the swab provides a substrate for the mold to adhere to. The swab sample is then removed from the growth medium after 24 hours for most samples and placed in a buffer solution. The sample is then subjected to chemical additives and/or mechanical forces to shear the hyphal strands. The sample is filtered, diluted, stained with a fluorochrome, counterstained with a quenching agent and analyzed on a Micro PRO™ flow cytometer. PCP materials containing microbe contaminants have fluorescence and scatter signals that indicate a compromised material.

### Example 1

More specifically, Example 1 represents the difference between a positive microbe sample (at a low spike number) and a negative sample, which is the same as the positive minus the microbe spike, for more than one microbe class. This example includes GILLETTE Series Shave Gel, Ultra moisturizing with glycerin. The growth medium used was Tryptic Soy Broth at a concentration of 30 g/L; soy lecithin at a concentration of 5 g/L, Tween-20 at a concentration of 40 mL/L; malt extract at a concentration of about 10 g/L; and dextrose at a concentration of about 10 g/L. Ten grams shave gel were added to 90 mL GEM (1:10) and neutralized 30 minutes. Four tubes, each with a swab placed in 19 mL growth enhancement media, were prepared. 1 mL 1:10 shave gel was added to all four tubes. A bacteria spike (<100 cfu S. aureus) was added to one tube, a yeast spike (<100 cfu C. albicans) was added to a second tube, a mold spike (<100 cfu A. niger) was added to a third tube, and no spike was added to a fourth tube to serve a product control. All tubes were then placed at 30° C.±2° C. on an orbital shaker for incubation. After 24 hours, the samples were removed from the incubator, the swabs were removed from their respective tubes and placed into individual sterile 15 mL conical tubes each containing 2 mL of phosphate buffer. The tubes were then subjected to a high vortex for 30 seconds and let sit 10 minutes for foam to settle. The samples were filtered through a 35 micron filter and diluted 1:10 in filtered phosphate buffer and analyzed on a flow cytometer under a method that uses 2× nucleic acid dye as the labeling agent with 1× Acid Black as the quenching component.

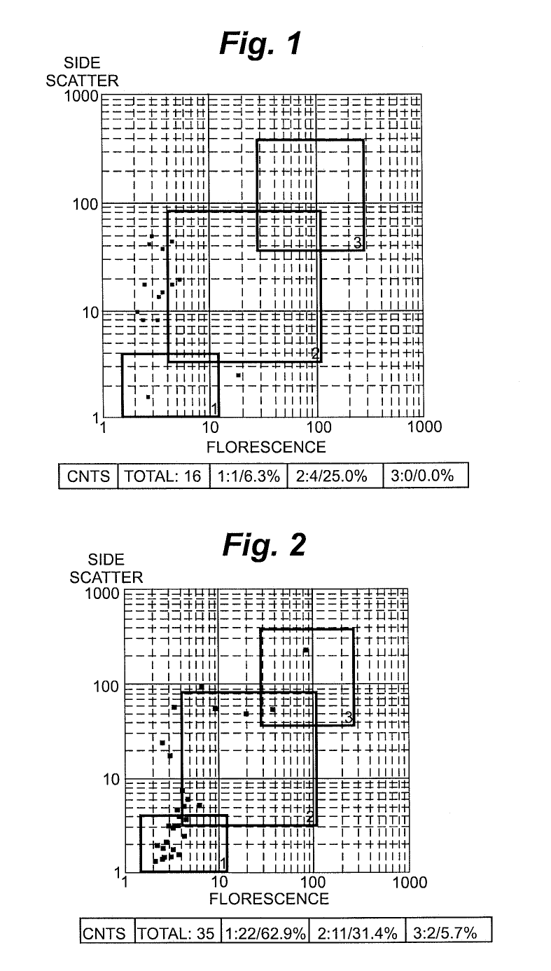

FIGS. 1-4 each show a plot of scatter intensity (y axis) versus fluorescence intensity (x axis) for an unspiked baseline sample (FIG. 1) and spiked samples (FIGS. 2-4) of shaving gel. The baseline plot of FIG. 1 shows background counts from the sample product matrix itself. As shown in FIGS. 2-4, the signals generated from the labeled microbes are shown in the several boxed regions of the plot of scatter signal versus fluorescence signal (a so-called scatter plot). The points in these plots not found in the baseline FIG. 1 plot correspond to microbes passing through the laser beam in a flow cytometry measurement. The plot can be color coded to indicate the relative amount of particles present on a particular coordinate of the plot. The number of such points can be related to the presence of microbes in the original sample. Test samples are considered positive when the signal to noise ratios of a boxed sample area meet or exceed a value determined by a product matrix or sample type.

FIG. 1 shows the type of measurement used to detect microbes in such an analysis. In the example shown in FIG. 1, the actual counts are represented in the scatter plot. As shown, three boxed areas are defined. Area 1, the mold area definition, is defined as the square located at the lower left of the plot. Area 2, the bacteria area definition, is defined as the middle square. Area 3, the yeast area definition, is defined as the square located in the upper right of the plot. The results are shown in Table 1:

TABLE 1
Product control (non-spiked)

| Mold | Bacteria | Yeast | | | | |
|---|---|---|---|---|---|---|
| counts/ | counts/ | counts/ | Mold | Bacteria | Yeast | Overall |
| mL | mL | mL | Result 1 | Result 2 | Result 3 | Result |
| 10 | 43 | 0 | Pass | Pass | Pass | Pass |

As shown, the sample passed the presence/absence test for all three detected microbe classes.

FIG. 2 shows the same sample as that shown in FIG. 1, except the sample has been spiked with A. niger mold. Table 2 summarizes the results:

TABLE 2
Mold (A. niger) spike

| Mold | Bacteria | Yeast | | | | |
|---|---|---|---|---|---|---|
| counts/ | counts/ | counts/ | Mold | Bacteria | Yeast | Overall |
| mL | mL | mL | Result 1 | Result 2 | Result 3 | Result |
| 241 | 120 | 21 | Fail | Ambiguous | Fail | Fail |

As shown, the spiked sample failed the mold presence/absence test, had an ambiguous result for the bacteria presence/absence test, and failed the yeast presence/absence test.

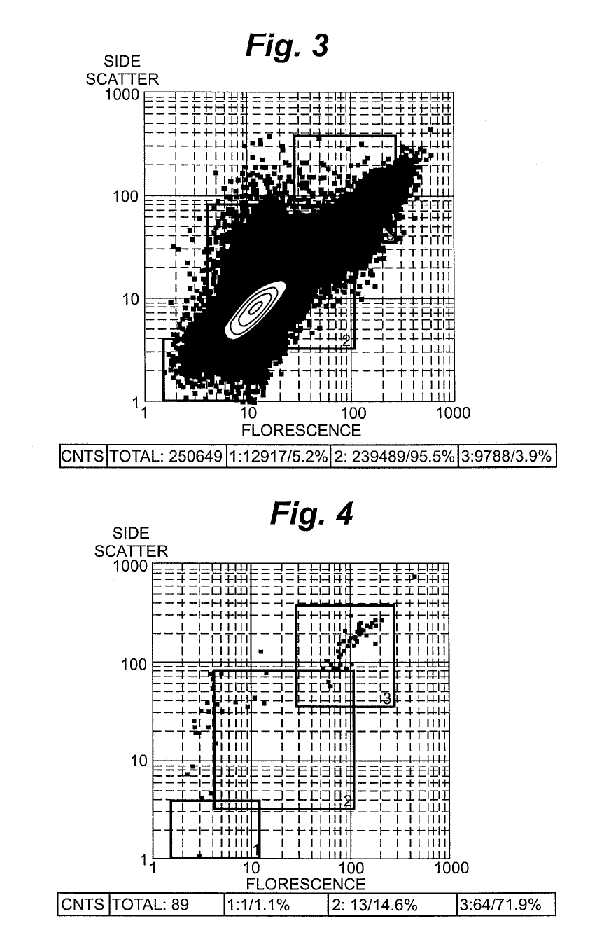

FIG. 3 shows the same sample as that shown in FIG. 1, except the sample has been spiked with S. aureus bacteria. Table 3 summarizes the results:

TABLE 3
Bacteria (S. aureus) spike

| Mold | Bacteria | Yeast | | | | |
|---|---|---|---|---|---|---|
| counts/ | counts/ | counts/ | Mold | Bacteria | Yeast | Overall |
| mL | mL | mL | Result 1 | Result 2 | Result 3 | Result |
| 142086 | 2634378 | 107667 | Fail | Fail | Fail | Fail |

As shown, the spiked sample failed the mold presence/absence test, failed the bacteria presence/absence test, and failed the yeast presence/absence test.

FIG. 4 shows the same sample as that shown in FIG. 1, except the sample has been spiked with C. albicans yeast. Table 4 summarizes the results:

TABLE 4
Yeast (C. albicans) spike

| Mold | Bacteria | Yeast | | | | |
|---|---|---|---|---|---|---|
| counts/ | counts/ | counts/ | Mold | Bacteria | Yeast | Overall |
| mL | mL | mL | Result 1 | Result 2 | Result 3 | Result |
| 10 | 142 | 703 | Pass | Ambiguous | Fail | Fail |

As shown, the spiked sample passed the mold presence/absence test, had an ambiguous result for the bacteria presence/absence test, and failed the yeast presence/absence test.

### Example 2

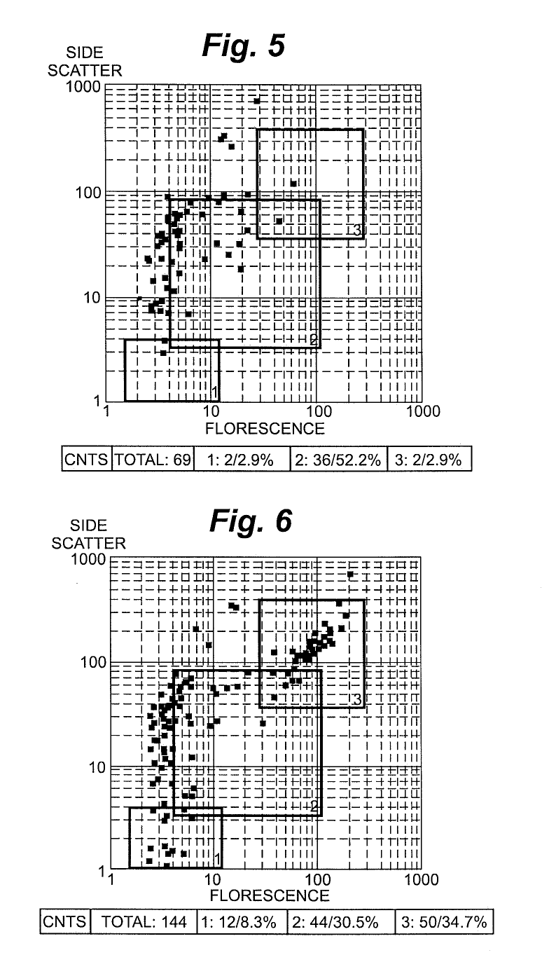

Samples of SUAVE Shampoo, Ocean Breeze Scent, were prepared and tested as in Example 1, except the samples were left in the incubator for 20 hours. FIG. 5 shows a plot of scatter intensity (y axis) versus fluorescence intensity (x axis) for an unspiked shampoo sample and FIG. 6 shows a plot of a shampoo sample spiked with C. albicans yeast and A. niger mold. As shown from the comparison of FIGS. 5 and 6, methods in accordance with embodiments of the invention are useful for simultaneously detecting the presence or absence of more than one microbe class.

Also by way of example and not limitation, a representative test method is provided below:
1. Warm 19 mL growth enhancement media (GEM) and substrate tubes to room temperature.
2. Aseptically prepare a product suspension be adding 10 g of product to 90 mL of phosphate buffer or GEM.
3. Mix the sample suspension to disperse the sample evenly.
4. Aseptically transfer 1 mL of product suspension to 19 mL of GEM in a tube containing a solid article (e.g., substrate).
5. Incubate at 30° C.±2° C., shaking (at approximately 200 rpm) overnight.
6. Remove substrate—usually around 24 hours—and place in 2 mL of phosphate buffer solution.
7. Vortex for about 15 seconds, and let tube rest for about 10 minutes.
8. Replace the standard cap on a tube with a 35 micron filter cap.
9. Filter 0.1 mL of the sample through the filter cap into about 2.9 mL of phosphate buffer solution, keeping the pipettor upright with the tip touching the filter membrane (this helps ensure the proper volume goes through the filter membrane)
10. Analyze on a Micro PRO™ using nucleic acid stain and/or Acid Black.
11. Compare results to a negative baseline. Anything greater than or equal to the predetermined positive cut off for the signal to noise ratio is considered positive for each microbe class.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations, which fall within the spirit and broad scope of the invention.

What is claimed is:

1. A method of simultaneously detecting the presence or absence of more than one microbe class in a sample, comprising:
applying a sample suspected of containing microbes to a growth medium;
associating the sample with one or more labeling agents; and
simultaneously detecting the presence or absence of the more than one microbe class in the sample by detecting the labeling agents.

2. The method of claim 1, wherein the sample includes a product selected from the group consisting of a personal care product and an over-the-counter medication.

3. The method of claim 1, wherein the labeling agents include a fluorescent agent.

4. The method of claim 3, wherein the fluorescent agent is selected from the group consisting of nucleic acid dyes, nucleic acid stains, fluorochrome labeled anti-mold antibodies, mold specific stains, fluorochrome labeled nucleic acid probes, and combinations thereof.

5. The method of claim 3, further including the step of adding a quenching agent after associating the sample with the fluorescent agent.

6. The method of claim 5, wherein the quenching agent comprises Acid Black.

7. The method of claim 1, wherein the growth medium comprises a nitrogenous substance, a sample neutralization agent, a carbon source, and a stimulation agent.

8. The method of claim 1, wherein a solid article is included with the growth medium, further comprising the step of removing the solid article from the growth medium.

9. The method of claim 8, further including the step of placing the solid article in a buffer solution.

10. The method of claim 1, wherein the sample is fragmented mechanically.

11. The method of claim 10, wherein the mechanically fragmenting step includes one or more steps from the group consisting of subjecting the sample to sonic energy, high shear force, and mechanical agitation.

12. The method of claim 11, wherein the sample is filtered after the mechanical fragmentation step.

13. The method of claim 1, wherein the detecting step includes placing the sample in a flow cytometer.

14. The method of claim 13, wherein the labeling agent includes a nucleic acid dye/stain selected from the group consisting of Syto 62, Syto 61, Syto 59, and Hexidium Iodide.

15. The method of claim 13, wherein the flow cytometer produces a scatter plot having separate predefined areas for bacteria, yeast, and mold.

16. The method of claim 15, wherein the scatter plot is compared to a scatter plot of a known substantially microbe-free sample to determine if the sample contains bacteria, yeast, or mold.

17. The method of claim 1, wherein the presence or absence of the more than one microbe class can be detected within 24 hours of the applying step.

18. A method of simultaneous detecting the presence or absence of more than one of bacteria, yeast and mold in a sample, comprising:
applying a sample suspected of containing more than one of bacteria, yeast, and mold to a growth medium, a substrate useful for supporting microbe growth included within the growth medium;
associating the sample with a labeling agent, the labeling agent including a fluorescent agent; and
simultaneously detecting the presence or absence of the more than one of bacteria, yeast, and mold in the sample by placing the substrate into a fluorescence chamber of a flow cytometer, the presence of the more than one of bacteria, yeast, and mold being detectable by its position on a scatter plot output of the flow cytometer.
